(19) United States
(12) Patent Application Publication
(10) Pub. No.: US 2002/0081602 A1
(43) Pub. Date: Jun. 27, 2002

(54) Method for the detection of compounds that modulate the effects of the obese (ob) protein

(75) Inventor: Lee James Beeley, Dorking (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.1.c.

(21) Appl. No.: 09/842,761

(22) Filed: Apr. 26, 2001

Related U.S. Application Data
(63) Continuation of application No. 09/297,442, filed on Jun. 18, 1999, now abandoned.
Application No. 09/297,442 is a 371 of international application No. PCT/GB97/02988, filed on Oct. 30, 1997.

(30) Foreign Application Priority Data
Nov. 1, 1996 (GB) 9622866.3
Nov. 1, 1996 (GB) 9622867.1
Nov. 1, 1996 (GB) 9622865.5
Nov. 1, 1996 (GB) 9622869.7
Nov. 1, 1996 (GB) 9622847.3
Nov. 1, 1996 (GB) 9622849.9
Nov. 1, 1996 (GB) 9622851.5
Nov. 1, 1996 (GB) 9622870.5
Nov. 1, 1996 (GB) 9622868.9
Nov. 1, 1996 (GB) 9622848.1
Nov. 1, 1996 (GB) 9622850.7

(51) Int. Cl. C12Q 1/68; G01N 33/574

(52) U.S. Cl. 435/6; 435/7.23

(57) ABSTRACT

A method for the detection of a compound that mimics, potentiates or inhibits the physiological effect of the ob-protein, which method comprises: (a) for a compound which mimics the physiological effect of the ob-protein, assessing the effect of the compound upon an ob-protein activated signal transducer and activator of transcription (STAT) DNA response element coupled to a reporter gene; or (b) for a compound which potentiates or inhibits the physiological effect of the ob-protein, assessing the effect of the compound upon the response provided by ob-protein upon an ob-protein activated STAT DNA response element coupled to a reporter gene; wherein, the response element and the reporter being expressed in an ob-protein response cell line, which cell line is selected from the list consisting of: a hypothalamic derived cell line; a pheochromocytoma derived cell line; a haematopoietic derived cell line; a pancreatic derived cell line; a liver derived cell line; a preadipocyte derived cell line; a skeletal muscle derived cell line; and an ovarian derived cell line; or the response element and the reporter are expressed in a cell line, which cell line (the engineered cell line) is also transfected with a polypeptide which is capable of mediating the stimulation by ob-protein of an ob-protein activated STAT DNA response element and contains the appropriate STAT proteins.

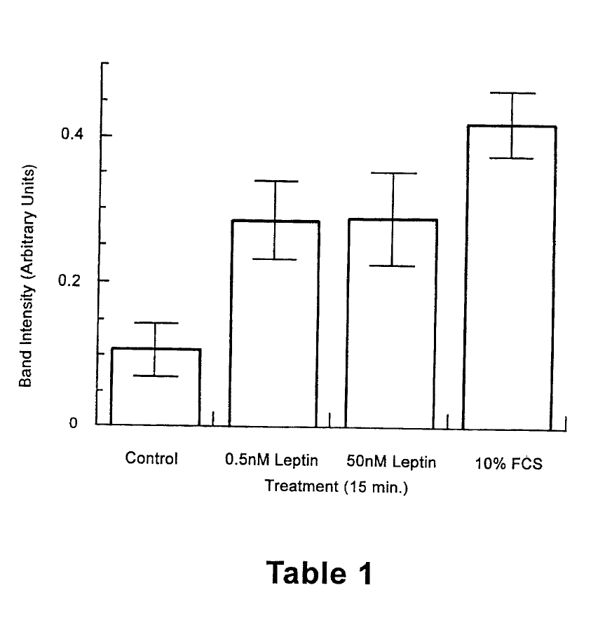

The invention relates to a novel method and more particularly to a method for the detection of compounds that mimic, potentiate or inhibit the physiological effects of the ob-protein.

The ob-protein (or leptin) is a secreted hormone that acts as signal from adipose tissue to other organs to regulate weight and energy balance (Zhang et. al., Nature, 1994, 372, 425). Additional roles for the ob-protein in haematopoietic and reproductive function have been suggested (Cioffi et. al. Nature Medicine, 1996, 2(5), 585). Protein molecules that contain a core composed of four α-helices forming a bundle of up-up-down-down topology comprise a family of cytokines and growth factors. Proteins of this family cause homo- and hetero-oligomerisation of membrane receptors known to activate kinase cascades resulting in gene transcription. Receptors of the family which are activated by oligomerisation fall into two broad classes; those such as epidermal growth factor receptor, which possess integral tyrosine kinase activity in their intracellular domains (A. Ullrich & J. Schlessinger, Cell, 1990, 61, 203-212), and those such as the IL4 and erythropoietin receptors, which lack this activity and mediate their response by way of an associated protein tyrosine kinase (J. N. Ihle et al., TIBS, 1994, 19, 222-227). Both receptor subtypes are activated by cytokines, but the 4-helix bundle proteins activate only the non-integral tyrosine kinase subtype. The non-integral protein tyrosine kinase receptors generally act through a pathway involving Janus kinase (JAK) and their associated signal transducers and activators of transcription (STAT) proteins. On activation STAT proteins bind to DNA response elements thereby controlling gene transcription. Oligonucleotide sequences comprising DNA regulatory elements of the general sequence TT(N)nAA have been identified (Seidel et al., Proc. Nat. Acad. Sci. USA., 1995, 92. 3041) as STAT response elements. These elements bind STAT proteins in response to signalling molecules such as cytokines.

In copending United Kingdom patent application number 9509164.1 we have described our discovery that the ob-protein is characterised by a four helix bundle tertiary structure. We now believe that the ob-protein interacts with a membrane bound receptor that activates a JAK-STAT kinase cascade and hence forms the basis for an assay system for the detection of compounds that mimic, potentiate or inhibit the physiological effects of the ob-protein. Such an assay has utility in selecting compounds for the treatment of weight, energy balance, haematopoietic, fertility and other disorders modulated by the ob-protein. The assay is especially useful for selecting compounds for the treatment of those disorders related to obesity, anorexia, cachexia and diabetes.

Copending International patent application number PCT/EP96/02291 relates to a novel detection method which uses JAK-STAT technology. We have now found a particularly advantageous detection method which also utilises this technology.

Accordingly, the invention provides a method for the detection of a compound that mimics, potentiates or inhibits the physiological effect of the ob-protein, which method comprises:

(a) for a compound which mimics the physiological effect of the ob-protein. assessing the effect of the compound upon an ob-protein activated signal transducer and activator of transcription (STAT) DNA response element coupled to a reporter gene; or

(b) for a compound which potentiates or inhibits the physiological effect of thee ob-protein, assessing the effect of the compound upon the response provided by ob protein upon an ob-protein activated STAT DNA response element coupled to a reporter gene;

wherein,

the response element and the reporter being expressed in an ob-protein responsive cell line, which cell line is selected from the list consisting of:

a hypothalamic derived cell line;

a pheochromocytoma derived cell line;

an haematopoietic derived cell line;

a pancreatic derived cell line;

a liver derived cell line;

a preadipocyte derived cell line;

a skeletal muscle derived cell line; and

an ovarian derived cell line; or

the response element and the reporter are expressed in a cell line, which cell line (the engineered cell line) is also transfected with a polypeptide which is capable of mediating the stimulation by ob-protein of an ob-protein activated STAT DNA response element and contains the appropriate STAT proteins.

A suitable cell line is a hypothalamic derived cell line.

A suitable cell line is a pheochromocytoma derived cell line.

A suitable cell line is an haematopoietic derived cell line.

A suitable cell line is a pancreatic derived cell line.

A suitable cell line is a liver derived cell line.

A suitable cell line is a preadipocyte derived cell line.

A suitable cell line is a skeletal muscle derived cell line.

A suitable cell line is an ovarian derived cell line.

Suitably, the response element and the reporter are expressed in a cell line, which cell line (the engineered cell line) is also transfected with a polypeptide which is capable of mediating the stimulation by ob-protein of an ob-protein activated STAT DNA response element and contains the appropriate STAT proteins.

An example of a hypothalamic derived cell line is the neuronal GTI-7 cell line described by W. C. Wetsel in Cellular and Molecular Neurobiology, 1995, 15(1), 43.

An example of a pheochromocytoma derived cell is the adrenal pheochromocytoma rat PC 12 cells (L. A. Greene and A. S. Tischler, Proc. Natl. Acad. Sci. USA. 1976, 73(7), 2424).

An example of an haematopoietic derived cell line is the HEL 92.1.7 (ATCC TIB 180) cell line derived from a human erythroleukemia (P. Martin, Science. 1982.216(4551), 1233).

A further example of an haematopoietic derived cell line is the K562(ATCC CCL-243) cell line derived from human chronic myelogenous leukemia (Proc. Soc. Exp. Biol. Med. 1981, 166, 546 and Blood, 1975, 45, 321).

An example of a pancreatic derived cell line is the BetaTC-3 cell line (T. J. Kieffer et al., Biochem. and Biophys. Res. Comm., 1996, 224, 552).

Further examples of pancreatic cell lines include RIN5mF cells, recently reported to respond to leptin (Md Shahidul Islam etal; Biochem Biophys Res Comm, 1997, 238, 851-855).

An example of a preadipocyte derived cell line is the 30A5 cell line (Y. Bai et al., J. Biol. Chem., 1996, 271(24), 13939).

Further examples of preadipocyte derived cell lines include differentiated 3T3-L1 and 3T3-F442A cell lines, both of which are commercially available.

An example of a liver derived cell line is the HepG2 cell line (B Cohen etal; Science, 1996, 274, 1185-1188 and Y Wang etal; J Biol Chem, 1997,272, 16216-16223) or H35 cell line (Y Wang etal; J Biol Chem, 1997, 272, 16216-16223).

Further examples of liver derived cell lines are WRL68 and Change liver cells, both of which are available commercially.

An example of a skeletal muscle derived cell line is the mouse myotube C2 C12 cell line (Nature, 1977, 270, 725).

An example of an ovarian derived cell line is the SK-OV-3 cell line. ATCC number HTB77, (J. Fogh and G Trempe in Human Tumour Cells In Vitro p155-159, J. Fogh (ed) Plenum Press, New York, 1975; J. Fogh and G Trempe J. Natl. Cancer Inst Bethesda, 1977, 587:209-214).

A suitable polypeptide which is capable of mediating the stimulation by ob-protein of an ob-protein activated STAT DNA response element is a functional isoform of the ob-gene receptor, for example that identified in Tartaglia et al., Cell, 1995, 83, 1263.

Suitably, the response element is coupled to a promoter gene, preferably a minimal promoter.

A suitable response element is a nucleotide of formula TT(N)ₙ AA, where N is any nucleotide and n is 4, 5 or 6.

A favoured response element is selectively activated by the intracellular events mediated the by the ob-protein interacting with its receptor. Such selective response elements can be determined by examining the relative activation of a range of response element-reporter gene constructs when transfected into an ob-responsive cell line by the ob-protein versus other cytokines.

A favoured response element is a nucleotide of formula TT(N)ₙ AA, where N is any nucleotide and n is 4 or 5.

A further suitable response element is TTCCCGGAA.

A further suitable response element is that region of the promoter of a gene regulated by the ob-protein that is required for STAT interactions. This gene will depend on the particular therapeutic use of the compounds to be selected by the assay.

A suitable reporter gene is firefly luciferase or chloramphenicol acetyltransferase enzyme.

A suitable promoter is a minimal promoter such as the herpes simplex virus thymidine kinase or SV40 promoter.

Other responsive cell lines can be identified using a displacement binding assay. Although binding may not be to a functional long form of the receptor, which is the form that transmits a signal to the cytoplasm. Identification of a functional long form of the receptor may be by PCR or Northern blot analysis (eg. Human ob-receptor: Tartaglia et al., Cell, 1995, 83, 1263). Ultimately responsive cells are detected by monitoring cellular events in the presence of varying concentrations of leptin. Potential methods for identifying candidate cell lines or monitoring these cellular events include the following:

1. Microphysiometer: This method detects small changes in pH resulting from biochemical changes in the cell. Ob-protein responsive cells upon stimulation may undergo biochemical changes that cause a small change in the extracellular acidification rate which can be detected by a silicon microphysiometer. The microphysiometer biosensor methodology has been reviewed by McConnell, Science, 1992, 257, 1906.

2. Electrophoretic mobility shift assay (EMSA): Nuclear extracts from cells after treatment with ob-protein are mixed with radiolabeled oligonucleotides containing a promiscuous or specific STAT response element DNA sequence. Extracts from cells that respond to the ob-protein may cause a gel shift of the oligonucleotide for the STAT response element.

References: Book “Recombinant DNA”, 2nd Edition, Watson et al., 1992, Page 158; Lamb et al., Blood, 1994, 83, 2063;

3. Measurement of protein phosphorylation assay: The coupling of receptor activation to the final response through tyrosine phosphorylation of intracellular proteins may be assayed by the use of antibodies recognising phosphorylated tyrosines. More specifically since the leptin receptor may stimulate tyrosine phosphorylation of the JAK/STAT pathway this method provides a method of detecting leptin response cell lines. Specific JAK/STAT antibodies may be used alongside antibodies for tyrosine phosphorylation to detect leptin activation in a leptin responsive cell line. Inhibition as well as stimulation of protein phosphorylation may occur. In particular, inhibition by the ob-protein of insulin stimulated phosphorylation of the insulin receptor and insulin receptor substrate-I has been shown in rat-1 fibroblasts over expressing insulin receptors (Kroder et. al 1996, Exp. Clin. Endocrinol. Diabetes, 104. suppl 2, p66)

4. Displacement binding: After incubation of cell lines with radiolabelled leptin, for example [¹²⁵I]-leptin, the non-specific binding versus specific binding of leptin can studied by the addition of unlabelled leptin. A high specific to non-specific ratio binding suggests that the cell line may contain the leptin receptor.

5. Detection of the protein for a functional form, preferably a functional long form, of the ob-receptor by use of selective antibodies.

6. Detection of mRNA for a functional form, preferably a functional long form, of the ob-receptor by Northern, RT-PCR or “slot blot” analysis.

7. Detection of increased c-fos mRNA after treatment with leptin. C-fos mRNA may be detected by Northern, RT-PCR or “slot blot” analysis.

Cell lines known to be involved in controlling aspects of the particular disease state for which compounds are being sought are preferred.

Cells lines derived from liver, brain, or pancreatic tissue and fibroblasts are particularly useful for “ob-responsive” cells for the assaying of compounds directed at obesity and diabetes. Certain areas of the brain are the focus of weight controlling and energy balance regulating effects of the ob-protein. The liver controls many metabolic processes that modulate lipid and glucose levels. Cells derived from particular regions of these organs containing the appropriate endogenous JAKs, STAT proteins and other intracellular proteins which are required for mediating the effects of the leptin are preferred.

The response element, the reporter, and preferably the promoter, are suitably incorporated into a vector capable of transfecting the ob-responsive cell line.

Suitable vectors are commercially available vectors, such as pGL2-basic luciferase vector (Promega).

A suitable configuration of the vector is the STAT DNA response element upstream of a promoter and a reporter gene. A more suitable configuration of the vector is the STAT DNA response element in multiple tandem repeats (2-10) upstream of a thymidine kinase promoter and a luciferase reporter gene.

Vectors are constructed containing a reporter gene for example firefly luciferase or chloramphenicol acetyltransferase enzyme linked to a minimal promoter for example the herpes simplex virus thymidine kinase or SV40 promoter. The DNA fragments for the STAT response element are inserted into the vector using appropriate restriction enzyme sites upstream of the minimal promoter.

The response element, the reporter and the promoter, as required, are incorporated into the vector using conventional expression techniques, for example the DNA fragments for the response element may be inserted into the vector using appropriate restriction enzyme sites upstream of the minimal promoter.

STAT response element-luciferase enzyme reporter systems can be constructed as described by Lamb et al.. Blood, 1994, 8, 2063 and Seidel et al., Proc. Nat. Acad. Sci USA., 1995, 92, 3041.

Ob-responsive cells are transfected with the STAT response element-minimal promoter-luciferase reporter constructs using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Potentiation or antagonist activity can be assayed by pre- or co-addition of an appropriate concentration of ob-protein to the compound under evaluation and measuring the potentiation or reduction in luciferase response relative to that of ob-protein alone. Standard methods exist for assaying luciferase enzyme activity for example Ow et al., Science, 1986, 234, 856 and de Wet et al., Mol. Cel Biol., 1987, 7, 725. as well as several commercial kits.

Stable cell lines can be generated by transfecting an “ob-responsive” cell line with the reporter construct and a selectable marker. Selectable markers are routinely used to generate stable cell lines as described in Recombinant DNA, 2nd edition, J. D. Watson et. al., 1992, page 216. These stably transfected cell lines can be used to generate high throughput assays for compounds that mimic, potentiate or block the physiological effects of the ob-protein.

The invention also extends to a compound that mimics, potentiates or inhibits the physiological effect of the ob-protein, when identified by the method disclosed herein.

The invention also extends to a kit of parts adapted for use in the method disclosed herein.

When used herein ‘a compound which mimics the physiological effects of the ob-protein’ refers to a compound which is capable of acting in the absence of the ob-protein to either stimulate the ob-protein receptor to provide substantially the same physiological effect as the ob protein or to activate a response down stream of this receptor (post-receptor).

When used herein ‘a compound that potentiates the physiological effect of the ob-protein’ refers to a compound which enhances the potency and/or maximal physiological effect of the ob-protein.

When used herein ‘a compound that inhibits the physiological effect of the ob-protein’ refers to a compound which reduces or substantially blocks the physiological effect of the ob protein.

The cDNA encoding the functional form of the polypeptide can be transfected under the control of a constitutive promoter, (eg a viral promotor) or a regulatable promoter to optimise the expression of the polypeptide for the identification of agonists or antagonists as necessary. Alternatively, the response element and the reporter are expressed in a cell line, wherein a constitutive or regulatable promoter has been engineered into a position upstream of the chromosomally encoded gene for the ob-protein recepter by the method of homologous recombination. Such methods are reviewed by Waldman, Critical Reviews in Oncology/Hematology, 1992, 12, 49 and a particular example is given in Riele et al. Proceedings of the National Academy of Sciences, 1992, 89, 5128.

The following examples illustrate the invention but do not limit it in any way.

### EXAMPLE

General Procedure:

Ob-responsive cells are transfected with a reporter plasmid containing a STAT response element, in multiple tandem copies upstream of a minimal promoter for example herpes simplex thymidine kinase and a luciferase gene reporter construct using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist activity can be assayed by pre- or co-addition of an appropriate concentration of ob-protein to the compound under evaluation and measuring the reduction in luciferase response relative to that of ob-protein alone. Standard methods exist for assaying luciferase enzyme activity for example Ow et al., Science, 1986, 234, 856 and de Wet et al., 1987, 7, 725. as well as several commercial kits.

### Example 1

Gt1-7 cells (W. C. Wetsel, Cellular and Molecular Neurobiology, 1995, 15(1), 43) are transfected with a reporter plasmid, pGL2-basic luciferase vector (Promega) containing an insert of an oligonucleotide corresponding to a four fold tandem repeat of the STAT response element, TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist activity can be assayed by pre- or co-addition of an appropriate concentration of ob-protein to the compound under evaluation and measuring the reduction in luciferase response relative to that of ob-protein alone. Standard methods exist for assaying luciferase enzyme activity for example Ow et al., Science, 1986, 234, 856 and de Wet et al., Mol. Cell Biol., 1987, 7, 725. as well as several commercial kits.

### Example 2

Rat PC12 cells (L. A. Greene and A. S. Tischler, Proc. Natl. Acad. Sci. USA. 1976, 73(7), 2424) are transfected with a reporter plasmid, pGL2-basic luciferase vector (Promega) containing an insert of an oligonucleotide corresponding to a four fold tandem repeat of the STAT response element. TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 3

HEL 92.1.7 (ATCC TIB 180) cells derived from a human erythroleukemia (P. Martin, Science. 1982, 216(4551), 1233) are transfected with a reporter plasmid, pGL2-basic luciferase vector (Promega) containing an insert of an oligonucleotide corresponding to a four fold tandem repeat of the STAT response element, TTCCCGGAA. upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 4

K562 (ATCC CCL-243) cells derived from human chronic myelogenous leukemia (Proc. Soc. Exp. Biol. Med. 1981, 166, 546 and Blood, 1975, 45, 321) are transfected with a reporter plasmid, pGL2-basic luciferase vector (Promega) containing an insert of an oligonucleotide corresponding to a four fold tandem repeat of the STAT response element, TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 5

The insulinoma BetaTC-3 cells (T. J. Kieffer et al., Biochem. and Biophys. Res. Comm., 1996, 224, 552) are transfected with a reporter plasmid, pGL2-basic luciferase vector (Promega) containing an insert of an oligonucleotide corresponding to a four fold tandem repeat of the STAT response element. TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase. and if appropriate β-galactosidase, activity. Antagonist and luciferase enzyme activity can be assayed as discussed above.

### Example 6

Preadipocyte derived 30A5 cells (Y. Bai et al., J. Biol. Chem., 1996, 271(24), 13939) are transfected with a reporter plasmid, pGL2-basic luciferase vector (Promega) containing an insert of an oligonucleotide corresponding to a four fold tandem repeat of the STAT response element, TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 7

Mouse myotube C2 C12 cells (Nature, 1977, 270, 725) are transfected with a reporter plasmid, pGL2-basic luciferase vector (Promega) containing an insert of an oligonucleotide corresponding to a four fold tandem repeat of the STAT response element, TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 8

Ovarian SK-OV-3 cells, ATCC number HTB77, (J. Fogh and G Trempe in Human Tumour Cells In Vitro p155-159, J. Fogh (ed) Plenum Press, New York, 1975; J. Fogh and G Trempe J. Natl. Cancer Inst Bethesda, 1977. 587:209-214) are transfected with a reporter plasmid, pGL2-basic luciferase vector (Promega) containing an insert of an oligonucleotide corresponding to a four fold tandem repeat of the STAT response element. TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 9

Hepatoma derived cell lines transfected with the functional form of the leptin receptor (Baumann et al., Proc. Nat. Acad. Sci., 1996, 93, 8374-8378) are co-transfected with a reporter plasmid, pGL2-basic luciferase vector (Promega) containing an insert of an oligonucleotide corresponding to a four fold tandem repeat of the STAT response element, TTCCCGGAA, upstream of the minimal promoter for herpes simplex thymidine kinase (−35 to +10) using standard methodology for example the calcium phosphate method (Graham and Van Der Eb, Virology, 1973, 52, 456). To correct for differences in transfection efficiency, the cells can be co-transfected with a reference plasmid expressing β-galactosidase activity. After a period of transfection (12-24 hours) the cells are treated with varying concentrations of compound and then harvested and lysed. The lysates are assayed for luciferase, and if appropriate β-galactosidase, activity. Antagonist and luciferase enzyme activity can be assayed as discussed above.

### Example 10

WRL68 cells were grown to confluence in their standard culture medium in the simultaneous presence of 10% foetal calf serum. At confluence the cells were transferred to serum-free medium for a minimum of 18 hours (lowering the serum concentration may also produce similar results). Serum-starved cells were then treated with leptin or with serum (as positive control for c-fos upregulation) for varying time periods from 5 minutes to 2 hours. RNA was extracted from the cells and subjected to gel electrophoresis and northern blotting. The blotted RNA was then probed with a c-fos labelled probe to analyze the expression of c-fos, the data being normalised by analysing the expression of beta-actin. Leptin treatment significantly enhanced c-fos expression in WRL68 cells (see Table 1) as would the positive control serum, and hence is expected to activate STATs. Therefore WRL68 cells is expected to be suitable for identification of leptin mimetics. Other cell lines could also be identified via analysis of c-fos expression in response to leptin.

1. A method for the detection of a compound that mimics, potentiates or inhibits the physiological effect of the ob-protein, which method comprises:
(a) for a compound which mimics the physiological effect of the ob-protein assessing the effect of the compound upon an ob-protein activated signal transducer and activator of transcription (STAT) DNA response element coupled to a reporter gene; or
(b) for a compound which potentiates or inhibits the physiological effect of the ob-protein, assessing the effect of the compound upon the response provided by ob protein upon an ob-protein activated STAT DNA response element coupled to a reporter gene;
wherein,
the response element and the reporter being expressed in an ob-protein responsive cell line, which cell line is selected from the list consisting of:
a hypothalamic derived cell line;
a pheochromocytoma derived cell line;
an haematopoietic derived cell line;
a pancreatic derived cell line;
a liver derived cell line;
a preadipocyte derived cell line;
a skeletal muscle derived cell line; and
an ovarian derived cell line; or
the response element and the reporter are expressed in a cell line, which cell line (the engineered cell line) is also transfected with a polypeptide which is capable of stimulating an ob-protein activated STAT DNA response element and contains the appropriate STAT proteins.

2. A method according to claim 1, wherein the hypothalamic derived cell line is the neuronal GT1-7 cell line.

3. A method according to claim 1, wherein the pheochromocytoma derived cell is the adrenal pheochromocytoma rat PC12 cells.

4. A method according to claim 1, wherein the haematopoietic derived cell line is the HEL 92.1.7 (ATCC TIB 180) cell line derived from a human erythroleukemia or the K562(ATCC CCL-243) cell line derived from human chronic myelogenous leukemia.

5. A method according to claim 1, wherein the pancreatic derived cell line is the BetaTC-3 cell line or RIN5mF cells.

6. A method according to claim 1, wherein the preadipocyte derived cell line is the 30A5 cell line or differentiated 3T3-L1 or 3T3-F442A cell lines.

7. A method according to claim 1, wherein the liver derived cell line is the HepG2 cell line or WRL68 and Change liver cells.

8. A method according to claim 1, wherein the skeletal muscle derived cell line is the mouse myotube C2 C12 cell line.

9. A method according to claim 1, wherein the ovarian derived cell line is the SK-OV-3 cell line, ATCC number HTB77.

10. A method according to claim 1, wherein the polypeptide capable of stimulating an ob-protein activated STAT DNA response element is a functional isoform of the leptin receptor

11. A method according to claim 1, wherein the response element is coupled to a promoter gene, preferably a minimal promoter.

12. A method according to claim 1, wherein the response element is a nucleotide of formula TT(N)ₙ, AA, where N is any nucleotide and n is 4, 5 or 6.

13. A method according to claim 12, wherein the response element is TTCCCGGAA.


SEQUENCE LISTING (4 sequences)

SEQ ID NO: 1 - DNA, 8 bp, Homo sapiens
  unsure (3)(4)(5)(6): Wherein n can be a,c,t or g
ttnnnnaa

SEQ ID NO: 2 - DNA, 9 bp, Homo sapiens
  unsure (3)(4)(5)(6)(7): Wherein n can be a,c,t or g
ttnnnnnaa

SEQ ID NO: 3 - DNA, 10 bp, Homo sapiens
  unsure (3)(4)(5)(6)(7)(8): Wherein n can be a,c,t or g
ttnnnnnnaa

SEQ ID NO: 4 - DNA, 9 bp, Homo sapiens
ttcccggaa